(19) United States
(12) Patent Application Publication
(10) Pub. No.: US 2018/0147140 A1
(43) Pub. Date: May 31, 2018

(54) Treatment and Prevention of ENT Infections

(71) Applicant: Darryl Thomas Finn, Irvine, CA (US)

(72) Inventor: Darryl Thomas Finn, Irvine, CA (US)

(73) Assignee: Darryl Thomas Finn, Irvine, CA (US)

(21) Appl. No.: 15/365,820

(22) Filed: Nov. 30, 2016

(51) Int. Cl. A61K 9/00; A61K 33/40; A61K 33/30; A61K 31/192

(52) U.S. Cl. CPC A61K 9/0043; A61K 31/192; A61K 33/30; A61K 33/40

(57) ABSTRACT

The present invention teaches novel pharmaceutical compositions, dosing and treatment regimens for ENT infections, such as rhinosinusitis. The compositions can also be used by those without active rhinosinusitis to reduce and remove harmful germ dwelling and colonizing inside the nasal cavity and its surrounding passages, and prevent rhinosinusitis and other infections from happening. In one of the embodiment of the invention, the composition comprises of hydrogen peroxide, zinc chloride and sodium benzoate.

### BACKGROUND OF THE INVENTION

Among the sensory organs of the human body, the nose, eye, ear and mouth are the ones directly interacting with the outside world, and also the most prone to microbial infections. The nose is particularly prone for bacterial infection owing mainly to its unique anatomical environment. This area is a complex series of recessed, moist, air-filled cavities duplicated on either side of the face and divided by the nasal septum. It begins with relatively arid nostrils, followed by middle and upper nose cavity (medically their terms are anterior naris, middle meatus and sphenoethmoidal recess). The inner lining of the nose is mucosa tissue that contains serous and mucus glands and large numbers of mast cells. This mucosa extends to the openings leading to the sinuses as well as the Eustachian tube where it is continuous with the mucosa of the sinuses and middle ear, respectively. Xenobiotic access begins through the open nostril and its relatively arid interior nasal concha, progresses into the deeper middle concha connecting to the largest sinus cavity located against the cheek bone, the maxillary, following deeper via the superior concha connecting to the ethmoid sinus cavity located between the ridge of the nose and the eye and the sphenoid cavity located behind the nose between nose and eye, continuing further to connect to the frontal sinus cavity located above each eye. Extending from the paranasal sinuses the passages open to the eye tear duct and Eustachian tube leading to the inner ear and finally merging together at the top of the throat. The deeper into the nose, the warmer, moister and darker the environment becomes. Most germs thrive in upper recesses of the nose, beyond the reach of conventional topical antibiotics or antiseptics can reach, thus becomes a dark spot for conventional topical treatment.

One of the most common nasal infections is rhinosinusitis, a bacteria nasal infection causing the inflammation of a nasal sinus and the surrounding tissue. Subtypes include chronic rhinosinusitis, acute rhinosinusitis, rhino maxillary sinusitis, and rhino horn sinusitis. Patients experience symptoms of nasal blockage, yellow nasal discharge, nasal bleeding and pain in the forehead or between the eyes. Statistics from the United States Center for Disease Control (CDC) for 2014 report 29.4 million adults diagnosed with infections classified as chronic rhinosinusitis resulting in 11.7 million doctors visits⁽¹⁾. Rhinosinusitis is termed chronic when it lasts more than three months. If the survey was expanded to include cases of infections lasting less than three months (acute rhinosinusitis) the estimated number would be significantly higher. Also outside the CDC statistics are cases of persistent bacteria dwelling and over colonizing without detectable symptoms of infection. The aggregate estimates suggest between ten and twenty percent of the general population is affected with persistent bacteria, fungus or virus dwelling and colonizing in the sinus cavities and surrounding connective passages including ear canal, tear duct and throat. At this time, no clinically proven effective treatment exists to clear the paranasal sinuses of bacteria, virus or fungus. The compositions and products presented in this invention is the breakthrough in this area.

Paranasal microbes and fungus reside beyond reach of antibiotics and antiseptics that are applied at the nostril opening. The topical antiseptics and antibiotics simply cannot reach the reservoir of bacteria in the deep cavities, i.e. the sphenoid cavity and frontal sinus cavity located in middle meatus and sphenoethmoidal recess, respectively. Some antiseptics may be effective against these infections but are too toxic to be placed deeper in the paranasal sinuses, toxic because medication will enter the digestive system through the upper back of the throat where mucus drains from the nasal area. Various formulations of alcohols, such as Nozin® series products⁽²⁾, are one example of an antiseptic that results in gastrointestinal disturbances if introduced into the digestive system. Beyond toxicity, antiseptics often irritate delicate paranasal tissues.

Several available non-toxic treatments do reach deeper into the paranasal channels; these treatments use a variety of approaches including droppers, washes and sprays. The most widely used is saline solutions but recent government clinical studies conclude saline solution is ineffective against infection (see the study cited below). Another treatment is glucocorticoid agents delivered in the same way (and sometimes in pill form) but glucocorticolds, commonly called steroids, address only the inflammation caused by the infection and relies on the body to remove bacteria with a more open path for mucus, again the most recent government clinical studies conclude glucocorticolds are not useful for sinusitis and, by extension, paranasal infection⁽³⁾.

Oral antibiotics are in use but require many weeks of dosage resulting in an unacceptable treatment cost for the millions of cases and with a high recurrence rate of the original condition. Where an antibiotic fails to remove all bacteria it serves only to fortify the bacteria resistance against future treatment. The US National Library of Medicine PubMed Health—National Institute of Health—provides a report on the most recent clinical trials addressing chronic sinusitis in the publication “Systemic and Topical Antibiotics for Chronic Rhinosinusitis”. This report was initially issued on Sep. 29, 2015 and updated on Apr. 26, 2016. The researchers conclude, “We found very little evidence that oral antibiotics are effective in patients with chronic sinusitis.” A careful reading of the study reveals saline solutions and steroids were included in the treatments⁽³⁾.

Surgery is sometimes appropriate when nasal polyps or deviations block passages but this condition does not describe the most common cases of infection and is often cost prohibitive.

There is evidence that bacteria and viruses dwelling and colonizing in the nasal and paranasal areas are the source of infections spread in hospital settings and long-term care facilities. The most serious among these are deadly Methicillin-Resistant Staphylococcus Aureus (MRSA) bacteria. Studies showed many patients suffer grievous injury and sometimes succumb to infections acquired while being hospitalized and not from the original condition treated. A study in 2014 showed that over 70% of the health care personnel have bacterial overgrow in their nasal cavity⁽¹⁾. With or without the symptoms of sinusitis, germs and fungus in the sinuses may result in adverse quality of life conditions including chronic fatigue, mild headache, ear infections, constant sinus drainage, bad breath and adverse voice quality.

### SUMMARY OF THE INVENTION

This application is for novel compositions and products effective to reduce and remove harmful germs, including bacteria, fungi and viruses, in the nasal and paranasal sinuses and surrounding connective passages, especially where the dwelling and colonization of bacteria, fungi and viruses has caused inflammation and the blockage of ENT (ear, nose and throat) pathways, i.e. a condition called rhinosinusitis. Its subtypes include chronic rhinosinusitis, acute rhinosinusitis, rhino maxillary sinusitis, and rhino horn sinusitis. This application is also for novel compositions and products designed to act as a prophylactic therapy for the potential infections in the nasal and paranasal sinuses, and connecting ear and throat tissues.

This invention is topically applied compositions comprising of well tolerated anti-infectives (antibiotics, antiviral, anti-fungal, antiseptics and sanitizers). The compositions can be self-administered via the nasal openings through suitable applicators, such as nasal droppers and nasal sprayers. The compositions along with their applicators, dosing and treatment regime are designed to reach the recesses of the sinus cavities and their surrounding connective passages. There are two basic utilities of the compositions: one is to actively treat rhinosinusitis, the other is to act as a prophylactic treatment against potential ENT infections when no significant symptoms appear. The compositions are applied intra-nasally using applicable dosage forms, such as liquids, sprays, droplets, gels, creams, ointments or foams.

The compositions of this invention comprise of hydrogen peroxide, sodium benzoate, and optionally zinc ion antiseptics. These active ingredients are preferably, diluted in de-ionized purified water. Using the invented compositions with a well-designed treatment regimen, our studies show the products have effectively reduce and remove harmful germs in the nasal and paranasal cavities, and thus effectively alleviate, and even cure rhinosinusitis.

### DETAILED DESCRIPTION OF THE INVENTION

This invention is a topically applied composition comprising of well tolerated anti-infectives (antibiotics, antiviral, anti-fungal, antiseptics and sanitizers). Particularly, the composition of this invention comprises of hydrogen peroxide, sodium benzoate, and zinc ion antiseptic, and any combination thereof.

### (1) Hydrogen Peroxide

A recent study by Harvard Medical School reveals the body produces hydrogen peroxide as a defense against infection⁽³⁾. Food grade purified hydrogen peroxide has a reliable history of safe usage and can be found in toothpastes and mouthwashes to fend off potential infections. The composition of the instant invention contains hydrogen peroxide in the amount between 1.5% to 0.6% mg/ml, within the range of toothpastes and mouthwashes. Preferably, the concentration of hydrogen peroxide in the inventive composition is about 1.5%, 1.4%, 1.3%, 1.2%, 1.1%, 1.0%, 0.99%, 0.98%, 0.97%, 0.96%, 0.95%, 0.94%, 0.93%, 0.92%, 0.91%, 0.90%, 0.89%, 0.88, 0.87%, 0.86%, 0.85%, 0.80%, 0.75%, 0.70%, and 0.6% mg/ml.

### (2) Zinc Chloride

Zinc is a proven anti-viral. It is present in this product at concentrations between 1.10% to 0.20% mg/ml, Particularly, the composition of the instant invention contains zinc chloride in the amount around 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 1.05%, and 1.10% mg/ml.

The zinc ion used in the instant invention is far less than the level found in a single multiple vitamin pill. The maximal load of zinc ion through compositions of this invention is 8.76 mg, 30% less than the 11 mg of zinc per tablet listed in Vitamin pill Centrum Silver.

### (3) Sodium Benzoate

Sodium benzoate is widely used in food and pharmaceutical products as stabilizer to fend off bacteria antiseptic in our usage range is used as antiseptics, stabilizer and pH adjustor. The concentration of sodium benzoate in the present invention is from about 0.1% to about 0.01% mg/ml, much lower than the 647 mg per kg of body weight, the value classified as generally regarded as safe (GRAS) by FDA.

### (4) pH Value

The composition of the instant invention has a pH value about 6.27 to about 7.05, preferably 6.27 to 6.40, a value in line with the undisturbed nasal pH.

Our invention is described above in general terms to allow the reader to understand the underlying principles, ideation, and problem solving. However, the description submitted in this patent application, including submitted drawing and other supporting documents, should not be interpreted as the entire scope, breadth, depth, or evolution of the problems, ideas, solutions, and descriptions captured in this document. Alternative practices or procedures utilizing the same principles and ideation are also covered in the invention disclosed herein.

The invention will now be explained with reference to a number of non-limiting examples.

### Examples

### Composition Example

FOUR (4) nasal compositions are exemplified below for reflection of the embodiments of the instant invention.

| | Composition 1 | Composition 2 | Composition 3 | Composition 4 |
|---|---|---|---|---|
| Hydrogen peroxide | 92.0 | 94.1 | 94.0 | 92.0 |
| (mg) | | | | |
| Zinc chloride (mg) | 60.0 | 84.0 | | 60.0 |
| Sodium benzoate (mg) | 5.0 | 5.0 | 5.0 | 5.0 |
| Glycerin | | | | 89 |
| De-ionized water 100 | 100 | 100 | | |
| q.s. (ml) | | | | |
| pH | 6.33 | 6.27 | 6.74 | |
| Dosage form | Nasal drop | Nasal drop | Nasal drop | Nasal ointment |

### Treatment Regime Example

For people with active rhinosinusitis, the typical treatment cycle is fourteen 14 days, the maximum usage.

Composition 1 can be used, and one application each day in each nostril of 7.3 milliliters per nostril will result in the application of

Sodium Benzoate—0.73 milligrams (0.05*7.3*2)
Hydrogen Peroxide—13.40 milligrams (0.92*7.3*2)
Zinc Chloride 8.76 milligrams (0.6*7.3*2)

Suitable applicators would be nasal droppers with slim enough tip to be partially inserted into the nasal cavity. A two ounce volume (about 60 milliliters) droppers designed by this invention is the perfect applicator. Two of these droppers would deliver the 14 days dosage.

After the sinusitis symptoms have under control, patient can switch to a maintenance and prophylactic daily dose. Composition 1 still can be used, but in a much less volume. For example, one application in each nostril each day can be reduced to 3.6 milliliters, about half of the 7.3 milliliters used by active sinusitis patients. In this way, each day, the patient will be exposed to

Sodium Benzoate—0.36 milligrams (0.05*3.6*2)
Hydrogen Peroxide—6.62 milligrams (0.92*3.6*2)
Zinc Chloride—4.32 milligrams (0.06*3.6*2)

Suitable applicators for this scenario can be either nasal droppers or nasal pump sprays. For example, an one ounce pump spray bottle can delivery about eight (8) dosage of this treatment regime.

For health care givers and those working in the hospital or other germ crowding settings, a maintenance dose can be applied at will on a weekly interval, but could be set to every other day or daily. For example, Composition 1 can be used with 3.6 milliliters per nostril. In here either nasal droppers or pump sprays can be used, but sprays are preferred due to ease of use.

### Patient Examples

### Patient Case 1

Caucasian male 62 years old in good health, whose daily med profile includes
Taking one statin generic drug daily
Taking one over the counter multiple vitamin daily
On average two medical exams each year
No diagnosed medical conditions or concerns

Kaiser patient uninterrupted care since 1997

Symptoms—blocked nasal passages especially on the right side of the face, continuous hardened yellow translucent pus discharge from right nostril, minor periodic bleeding, nasal sound quality of voice, a propensity for cold virus to linger and a whistle sound while breathing through the nostrils.

A formal diagnosis of chronic sinusitis from Kaiser Permanente of California occurred January 2001 with a symptom history suggesting infection since early adulthood. The Kaiser treatment program consisted of a saline rinse bulb and recommendation of daily saline sinus rinsing along with a nasal steroid spray available over the counter. For four months the patient followed the regimen of daily saline rinsing and steroid nasal spray resulting in no relief of symptoms. The patient continued occasional use of the regimen over the following 15 years with no noticeable improvement.

In 2009 a CT scan was taken (x-ray cross sectional tomographic imagery). The image revealed no polyps or deviated septum but showed the right maxillary sinus cavity completely blocked and filled with fluid.

This patient's condition remained unchanged until March 2016 when treatment therapy described in this patent application began.

It is interesting to note during the many years of sinusitis the patient completed prescribed antibiotic treatments for other purposes—dental infections on four occasions, one urinary tract infection and one surgery to remove impacted wisdom teeth. The patient reported no relief from the sinusitis as a result of the oral antibiotics—amoxicillin was most frequently used.

### Treatment Using the Invention Regime

Treatment began on Mar. 9, 2016 with a new and unique product. The formulation used is Composition 1 in the Formulation Example.

Initial treatment was for 14 days, one dose daily in each nostril, approximately ¼ ounce (7.3 milliliters) administered with a nose dropper while the patient was lying down with chin inclined upward—sometimes with head over the edge of bed or pillow to increase the incline of the forehead to be lower than the chin. The patient remained unmoving for five minutes with no blowing of the nose to allow gravity saturation to reach the deep passages. The dosage was taken just before sleeping to encourage a deeper flow as the patient slept.

After day 3 the patient reported no relief of symptoms but reported the onset of a strong bitter taste and continuous flow of mucus throughout the day especially when bending the head below the waist during daily activities. The patient reported the same experience for days 4, 5, 6, 7 with the phenomenon gradually diminishing around day 8 or 9 but continuing at reduced levels for approximately 20 days. Beginning day 9 the patient expressed new sensation of lightness and clear breathing and a reduction of facial pressure that was apparently present but ingrained for such a period of time as to have reduced body awareness. The patient reported heightened awareness of, and a sensation in, what seemed to describe the large maxillary sinus cavity.

Beginning day 15 the method of delivery was altered, the dropper was no longer used and instead a plastic bottle with misting pump was used to deliver smaller doses of approximately ⅛ ounce (about 3.6 milliliters) into each nostril while the patient was vertical and then immediately assumed the lying position with chin up—again just before sleeping for the night. This regimen was continued for 6 days.

No treatment was given on days 20, 21 and 22. Moving forward the regimen was one application of the nasal spray followed by 3 days of no application and continuing in the manner for the 30 days.

After day 50 the product was delivered using the spray once every 7 days at the same dose of 3.6 milliliter (⅛ fluid ounce)

From day 51 to day 195 (time of application) the patient has self-administered the spray approximately once each week.

### Assessment

The patient reports using a simplified Health Related Quality of Life Scale of from 0 to 9 with 5 representing the condition at the beginning of the treatment period, zero a total absence of the condition and 9 a dramatic increase in severity of the condition.

| HRQL | Treatment Period Date |
|---|---|
| 5 | Mar. 9, 2016 - treatment begins |
| 3 | Apr. 10, 2016 |
| 2 | May 10, 2016 |
| 1.5 | Jun. 10, 2016 |
| 1.5 | Nov. 10, 2016 |

The patient kept a diary of contemporaneous comments during the treatment and includes the following.

“Near the end of the first week I experienced a heavy flow of mucus down the back of my throat, it was continuous and with an unusual foul taste. The taste and flow became less noticeable after that but was still occurring. At about three weeks there was an increase in the flow and foul taste exactly as before but less than the first time. After four or five days this increase also began to diminish.

Day 30 and the mucus flow and foul taste were gone.”

### Patient Self Evaluation

After more than eight months the patient still rates his condition as having improved from the original HRQL score of 5 to the present 1.5.

The patient reports no flow of pus or bleeding from his right nostril, a sense of light openness in both nasal sinus passages and an absence of pressure on the right maxillary sinus cavity, an absence of nasal whistling sounds, free open breathing through the nose (particularly when lying down) and a general improvement in well-being possibly owing to the relaxed immune system.

The patient currently reports HRQL of 1.5 and has had the same assessment for the past 5.5 months. This HRQL level represents the patient's continuous sensation of an open but not always fully open sensation of his entire sinus nasal system. The patient did report a period of 3 weeks in month 5 (July) during which no medication was used and a sense of regressing to a less open flow of air but not to any other previous symptoms.

The patient reports no sense of burden to the ongoing maintenance use of the spray a few nights each month. The patient does continue to express a feeling of relief and gratefulness for his improved quality of life.

### Observations and Notes

It may be the patient's initial report of heavy flow and foul taste signaled a significant load of medication had accumulated in the interior of the right maxillary sinus cavity—the one shown fully closed, full of fluid and under pressure by the 2009 CT scan. The interaction of the h2o2 with skin tissue is predicted to loosen the tissue surface. This would separate the tissue surface from bacteria and mucus. Concurrent with that process the anti-bacterial and anti-fungal qualities of h2o2 in combination with sodium benzoate would be killing the bacteria, virus or fungus and set in motion its decay. This, in turn, would reduce infectious swelling that had pinched and held the cavity closed at its opening into the concha passages. With the maxillary cavity now filled with fluid of dead and dying bacteria/fungus/virus and the cavity now open, a sudden outflow would occur—an outflow into the middle and superior concha and into the top of the throat and back of the tongue where the foul taste would manifest. It is likely this same process occurred in the smaller ethmoid, sphenoid and frontal sinus cavities as each one opened in turn and as the concha passages linking them gradually and progressively opened more deeply. This progression of emptying the cavities first large then smaller would parallel the timeline of higher and lower mucus flow and foul tastes reported by the patient.

The patient's ongoing rating of 1.5 is predicable because of the nature of chronic sinusitis. Chronic sinusitis sufferers have irreparable damage to the paranasal linings. This is the very condition that makes chronic sinusitis a life long condition prone to continuous infection; even healthy mucus secretions do not flow smoothly through the damaged areas.

While the patient's self assessment is subjective it nevertheless far exceeds the clinical trial results reported by the US National Library of Medicine PubMed Health publication in the article “Systemic and topical antibiotics for chronic sinusitis” Sep. 29, 2015 from the Cochrane Database of Systematic Reviews where on page 1 Author's Conclusions we find that after multiple studies using oral and topical antibiotics “The size of the improvement was modest (0.5 on a five point scale) and only seen at the end of three month treatment; by three months later no difference was found”. This clinical trial is the most recent and comprehensive study available to address paranasal infections. In summary, the study used every known available treatment and concluded after six months the treatments resulted in no improvement in the condition and no improvement in the patient self-assessment—health related quality of life (HRQL). In conclusion all current therapies were a total failure. The medicines included, saline rinses, oral antibiotics, topical antibiotics, topical and oral steroids.

### Patient Case 2

Caucasian male 61 years old, whose daily med profile includes
Active prescriptions—statin generic, Wellbutrin, sildenafil citrate
Taking one over the counter multiple vitamin
On average one medical exam yearly
A recent mental stress diagnosis related to a pending divorce
A diagnosis of fatigue, elevated blood pressure and over weight

Symptoms—blocked nasal passages, sinus headaches, continuous discharge of mucus from both nostrils, postnasal discharge, minor periodic bleeding, and pressure sensation around eyes.

Composition 1 and the same treatment regime was used as with case study 1.

| HRQL | Treatment Period Date |
|---|---|
| 5 | Jun. 12, 2015 - treatment begins |
| 4 | Jul. 11, 2015 |
| 3 | Aug. 11, 2015 |
| 2 | Sep. 15, 2015 |
| 2 | Dec. 8, 2015 |

Based on the patient's own recount, most of nose and headache symptoms were gone around September, by December, pressure sensation around eyes are also relieved. Patient self-evaluated as cured by this regime.

### Patient Case 3

Asian female 46 years old in general good health, with only daily vitamin supplements intake. She complaint about mild nasal blockage and pressure sensation around eyes for 3 months since February of 2016. Doctor diagnosed as negative in sinusitis, but suspected bacteria or viral overgrown in the nasal area. Patient decided to self-administer Composition 3 daily starting late July, with about 4.0 milliliter in each nostril using a pump spray. Symptoms totally alleviated in early October, but still randomly administer Composition 3 till now. No abnormality or side effects reported by the patient, only confirming clear breathing and lightened feel in the upper face area.

### REFERENCES

1. CDC publication “Chronic Sinusitis”, www.cdc.gov/getsmart/antibiotic-use/URI/sinus-infection.html, retrieved on Oct. 18, 2016
2. “NozinPro Health Professional Resource”, www.nozinpro.com, retrieved on Nov. 21, 2016.
3. “Systemic and topical antibiotics for chronic rhinosinusitis”, Head K, Chong L Y, Piromchai P, etc. Cochrane Database Syst Rev., 2016 Apr. 26; 4: CD011994.
4. “Hydrogen peroxide marshals immune system”, HMS Press Release, Harvard Medical School, Office of Communications and External Relations, published online on Jun. 3, 2009, retrieved on Oct. 17, 2016
5. “Antiviral activity of zinc salts against transmissible gastroenteritis virus in vitro”, Wei Z1, Burwinkel M, Palissa C, Ephraim E, Schmidt M F., Vet Microbiol. 2012 Dec. 7; 160(3-4):468-72. doi: 10.1016/j.vetmic.2012.06.019. Epub 2012 Jun. 26.
6. “Zinc and the special senses”, Russell R M, Cox M E, Solomons N., Ann Intern Med. 1983 August; 99(2):227-39.
7. CFR—Code of Federal Regulations Title 21, http://www.accessdata.fda.gov/scripts/cdrh/cfdocs/cfcfr/CFRSearch.cfm?fr=184.1733, retrieved on Oct. 18, 2016.

1. A method for the treatment of rhinosinusitis by topical application of a composition comprising of hydrogen peroxide and sodium benzoate.

2. The method of claim 1, wherein the concentration of hydrogen peroxide is no more than about 3% mg/ml and sodium benzoate is no more than about 0.1 mg/ml.

3. The composition of claim 1, further comprising a zinc ion containing anti-infective.

4. The composition of claim 3, wherein the zinc ion containing anti-infective is selected from zinc oxide, zinc dioxide, zinc sulfate, zinc chloride, zinc citrate, zinc phenol sulfonate, zinc stearate, and zinc undecylenate.

5. The composition of claim 4, wherein zinc ion containing anti-infective is zinc chloride and its concentration is between about 0.4% mg/ml to about 1.0% mg/ml.

6. The method of claim 1, wherein the composition comprises of about 0.92% mg/ml of hydrogen peroxide, about 0.05 mg/ml sodium benzoate, and about 0.6 mg/ml zinc chloride.

7. The method of claim 1, wherein the topical application is intra-nasal application through nasal dropper or nasal pump spray, or the combination thereof.

8. The method of claim 6, wherein the topical application is applied once daily for about 14 days, and with about 7.3 milliliter applied in each nostril.

9. The method of claim 8, wherein the topical application continued daily after the initial 14 days, but reduced the daily dose in each nostril from about 7.3 milliliter to about 3.6 milliliter.

10. A method for reducing and removing harmful germ dwelling and colonizing in nasal cavity of those without active rhinosinusitis symptoms by topical application of a composition comprising hydrogen peroxide; and sodium benzoate.

11. The method of claim 10, wherein the concentration of hydrogen peroxide is no more than about 1% mg/ml and sodium benzoate is no more than about 0.1 mg/ml.

12. The method of claim 11, wherein the composition containing about 0.94% mg/ml of hydrogen peroxide, about 0.05 mg/ml sodium benzoate.

13. The composition of claim 10, further comprising a zinc ion containing anti-infective.

14. The composition of claim 13, wherein the zinc ion containing anti-infective is selected from zinc oxide, zinc dioxide, zinc sulfate, zinc chloride, zinc citrate, zinc phenol sulfonate, zinc stearate, and zinc undecylenate.

15. The composition of claim 14, wherein zinc ion containing anti-infective is zinc chloride and its concentration is between about 0.4% mg/ml to about 1.0% mg/ml.

16. The method of claim 10, wherein the topical application is intra-nasal application through nasal dropper or nasal pump spray, or the combination thereof.

17. The method of claim 10, wherein the topical application is applied weekly, or every other day or daily with each nostril applied about 3.6 to about 4.0 milliliter of the composition.

18. The method of claim 17, wherein the topical application is intra-nasal application through nasal pump spray.
